(19) United States
(12) Patent Application Publication
(10) Pub. No.: US 2024/0262855 A1
(43) Pub. Date: Aug. 8, 2024

(54) A Method For Producing Cationic Saccharides

(71) Applicant: KEMIRA OYJ, Helsinki (FI)

(72) Inventor: Erkki Johannes METSÄLÄ, Espoo (FI)

(21) Appl. No.: 18/565,781

(22) PCT Filed: May 18, 2022
(86) PCT No.: PCT/FI2022/050332

§ 371 Date: Nov. 30, 2023

(30) Foreign Application Priority Data
Jun. 3, 2021 (FI) 20215645

(51) Int. Cl. C07H 15/04

(52) U.S. Cl. CPC C07H 15/04

(57) ABSTRACT

The present invention relates to a method for producing cationic saccharides by reacting saccharide with a compound having a conjugated electron withdrawing group via oxa-Michael addition reaction for producing a derivatized saccharide followed by cationizing the derivatized saccharide. The present invention further relates to use of the cationic saccharides as flocculants and as strength agents.

### TECHNICAL FIELD

The present disclosure generally relates to a method for producing cationic saccharides. The disclosure relates particularly, though not exclusively, to a method for producing cationic saccharides via oxa-Michael addition reaction.

### BACKGROUND

This section illustrates useful background information without admission of any technique described herein representative of the state of the art.

Flocculation is a water treatment process where solids form larger clusters, or flocs, to be removed from water. Flocculants are substances that promote agglomeration of fine particles present in a solution, creating a floc, which then floats to the surface (flotation) or settles to the bottom (sedimentation).

Flocculants can be organic or inorganic, and come in various charges, charge densities, molecular weights, and forms.

Organic polymeric flocculants are widely used, due to their ability to promote flocculation with a relatively low dosage. Although, their lack of biodegradability and the associated dispersion of potentially harmful monomers into water supplies is causing the focus to shift to biopolymers, which are more environmentally friendly. The problem with these is they have a shorter shelf-life, and require a higher dosage than organic polymeric flocculants. To combat this, combined solutions are being developed, where synthetic polymers are grafted onto natural polymers, to create tailored flocculants for water treatment that deliver the optimum benefits of both.

Paper strengthening agents play an important role in the papermaking industry with the increase of secondary fibre application. The most commonly used paper strengthening agents are polyacrylamide, starch, chitosan, and other polymers. Starch is currently the most widely used dry-strength agent because of its relatively low price and high performance. Starch has an abundance of hydroxyl groups, which form hydrogen bonds with wood fibres to improve paper strength. Starches are often modified with a cationic charge or with amphoteric starches to increase starch retention.

There is still a need for new methods for producing chemicals for water treatment and strength chemicals for papermaking.

### SUMMARY

The following presents a simplified summary of the features disclosed herein to provide a basic understanding of some exemplary aspects of the invention. This summary is not an extensive overview of the invention. It is not intended to identify key/critical elements of the invention or to delineate the scope of the invention. Its sole purpose is to present some concepts disclosed herein in a simplified form as a prelude to a more detailed description.

In a first aspect the present invention provides a method for producing cationic saccharide, comprising reacting a saccharide with a compound having a conjugated electron withdrawing group via oxa-Michael addition reaction in presence of a base or mixture of bases for producing a derivatized saccharide and cationizing the derivatized saccharide.

In a second aspect the present invention provides derivatized saccharides.

In a third aspect the present invention provides cationic saccharides.

In a fourth aspect the present invention provides a use of the cationic saccharide produced with the method of the present invention or the cationic saccharide of the present invention as a flocculant in industrial or municipal wastewater treatment, as a strength agent, a retention agent, a drainage agent or a sizing agent in papermaking, as a coagulant agent or as an additive in textile, cosmetic or paint industry.

It was surprisingly found that derivatized saccharides can be synthesized via oxa-Michael reaction in mild conditions. It was additionally found that by subjecting the derivatized saccharides to conventional cationization cationic saccharides can be produced. With the method of the present invention cationic saccharides can be produced with a simple method.

With the method of the present invention synthesis of biobased and biodegradable precursors of cationic polymers can be prepared via a route avoiding epoxide chemistry. The method of the present invention provides a route to environmentally-friendly polymers and cationic polymers, such as cationic saccharides.

It was also found that the produced derivatized saccharides and cationic saccharides can be used in variety of applications, for example, as flocculants in wastewater treatment and as a strength compounds in papermaking.

It was also surprisingly found that the smaller the molar mass of the cationic moiety bound to the saccharide via the ether bond the better results are obtained with said cationic saccharide in further applications. Examples of such applications are use of said cationic saccharide as a flocculant in industrial or municipal wastewater treatment, as a strength agent, a retention agent, a drainage agent or a sizing agent in papermaking, as a coagulant agent or as an additive in textile, cosmetic or paint industry.

Without wishing to bound to any theory, the better results are believed to be due to the ratio of carbon number in the cationic moiety to the ability of the electron withdrawing group to withdraw electrons, i.e. polar effect.

The appended claims define the scope of protection.

### BRIEF DESCRIPTION OF THE FIGURES

FIG. 1 shows 1HNMR profile of a reaction mixture comprising glucose(monohydrate), dimethylaminoethyl acrylate and a-d-glucose (C6-OH) derivatized with 2-(dimethylamino)ethyl acrylate.

FIG. 2 shows 13CNMR profile of a reaction mixture comprising glucose(monohydrate), dimethylaminoethyl acrylate and a-d-glucose (C6-OH) derivatized with 2-(dimethylamino)ethyl acrylate.

### DETAILED DESCRIPTION

In first aspect the present invention provides a method for producing cationic saccharides. More particularly, the present invention provides a method for producing cationic saccharides comprising reacting a saccharide with a compound having a conjugated electron withdrawing group via oxa-Michael addition reaction in presence of a base or mixture of bases for producing a derivatized saccharide, and cationizing the derivatized saccharide for producing the cationic saccharide.

By “Oxa-Michael addition reaction” is meant a conjugate nucleophilic addition reaction involving O-nucleophiles (Michael donors) and Michael acceptors.

By “an electron withdrawing group” is meant a group that draws electrons away from reaction center. Examples of electron withdrawing groups are halogens (F, Cl), nitriles (CN), carbonyls (RCOR′) and nitro groups (NO₂)

By “a compound having a conjugated electron withdrawing group” is meant a compound that has an electron withdrawing group that is conjugated; for example CH2=CH—C═O (i.e. double bond-single bond-double bond) and CH2=CH—NR2 (i.e. double bond-single bond).

In one embodiment the carbon-carbon double bond of the compound having a conjugated electron withdrawing group is terminal i.e. primary.

In one embodiment the carbon-carbon double bond of the compound having a conjugated electron withdrawing group is intramolecular.

In a preferred embodiment the carbon-carbon double bond of the compound having a conjugated electron withdrawing group is terminal i.e. primary.

In one embodiment a saccharide is reacted with a compound having a conjugated electron withdrawing group via oxa-Michael addition reaction by bringing the saccharide into contact with the compound having a conjugated electron withdrawing group in presence of a base or a mixture of bases.

The derivatized saccharide is formed via the oxa-Michael addition reaction. In oxa-Michael addition reaction carbon-carbon double bond of a compound having a conjugated electron withdrawing group reacts with hydroxyl group of the saccharide, thus, forming the derivatized saccharide.

In one embodiment one or more hydroxyl groups of the saccharide react with one or more of the compound having a conjugated electron withdrawing group, thus forming derivatized saccharide.

The derivatized saccharide can be cationized with any suitable method or reaction in the art. An example of such reaction is Menshutkin reaction. Menshutkin reaction converts a tertiary amine into a quaternary ammonium salt by reaction with an alkyl halide.

In one embodiment the derivatized saccharide is recovered prior the cationization.

In one embodiment the recovered derivatized saccharide is cationized.

In one embodiment the derivatized saccharide is recovered prior cationization and the recovered derivatized saccharide is cationized.

In one embodiment the saccharide comprises monosaccharides, disaccharides, oligosaccharides, polysaccharides or a mixture thereof.

In one embodiment the monosaccharide comprises glucose, fructose, galactose, mannose or a mixture thereof.

In one embodiment the disaccharide comprises sucrose, lactose, maltose or a mixture thereof.

In one embodiment the oligosaccharide comprises glycan, raffinose, maltodextrin, cellodextrin or a mixture thereof.

In one embodiment the polysaccharide comprises starch, glycogen, galactogen, cellulose, chitosan, chitin, guar gum, pectin, dextran, α-glucan, cyclodextrin such as ß-cyclodextrin or a mixture thereof.

In one embodiment the saccharide is glucose or cellulose.

In one embodiment the compound having a conjugated electron withdrawing group (EWG) comprises compounds having formula R1R2C═CR3-EWG, wherein R1, R2 and R3 represents independently a hydrogen atom or an alkyl chain having 1 to 20 carbon atoms; and EWG represents an electron withdrawing group preferably a halogen, a nitrile, a carbonyl or a nitro group.

In one embodiment the R1, R2 and R3 represent independently a hydrogen atom or an alkyl chain having 1 to 10 carbon atoms

In one embodiment the compound having a conjugated electron withdrawing group is cationic.

In one embodiment the compound having a conjugated electron withdrawing group is selected from one of the following compounds I-X

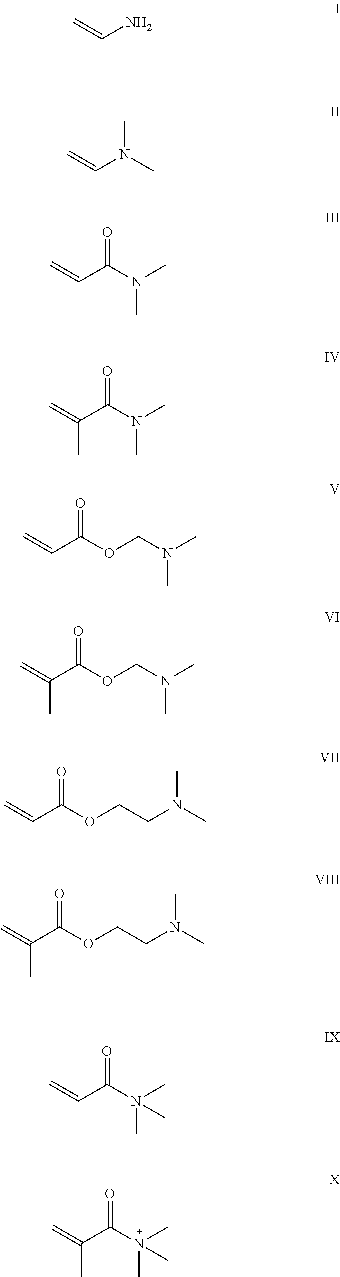

In one embodiment the compound having a conjugated electron withdrawing group is compound VII or VIII

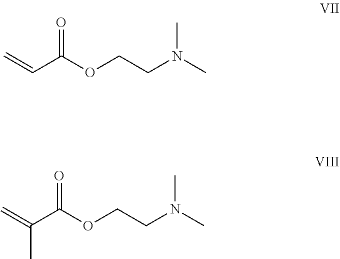

In one embodiment the compound having a conjugated electron withdrawing group is compound III or IV

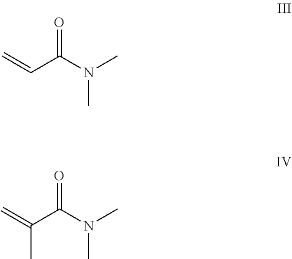

In one embodiment the compound having a conjugated electron withdrawing group is compound V or VI

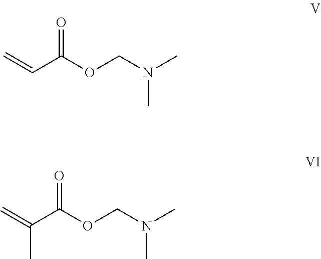

In one embodiment the compound having a conjugated electron withdrawing group is compound IX or X

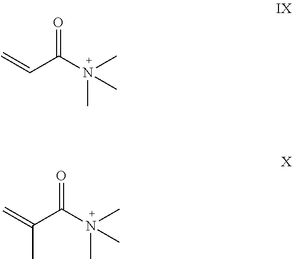

In one embodiment the compound having a conjugated electron withdrawing group is compound III, IV, V, VI, IX or X.

In one embodiment the compound having a conjugated electron withdrawing group is selected from one of the following compounds XXI-XXX

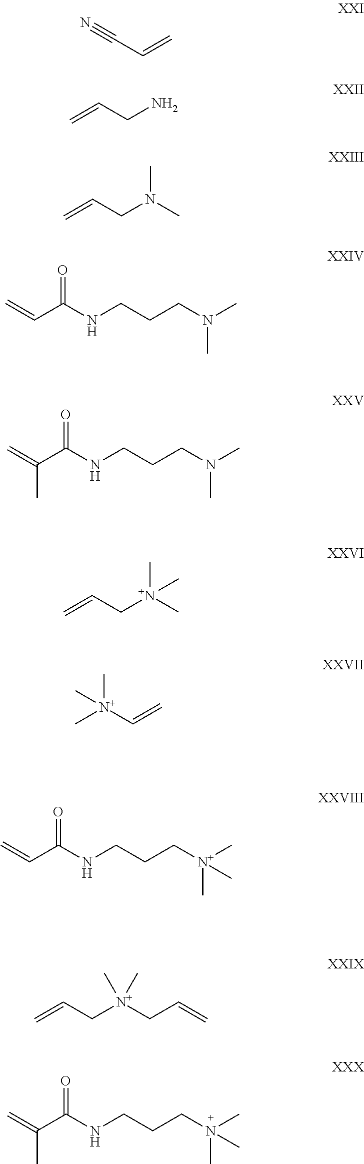

In one embodiment in the method for producing cationic saccharides mole ratio of the saccharide to the compound having a conjugated electron withdrawing group is 1:1.

In one embodiment the base present in the method for producing cationic saccharides comprises NaOH, KOH, potassium tert-butoxide (tBUOK), diazabicycloundecene (DBU), MeONa or a mixture thereof.

In one embodiment temperature in the oxa-Michael addition reaction is 15° C.-30° ° C., preferably 18° C.-25° C.

In one embodiment pressure in the oxa-Michael addition reaction is prevailing atmospheric pressure, preferably normal atmospheric pressure, more preferably about 1 bar.

In one embodiment the oxa-Michael addition reaction takes place in a liquid medium. Preferably the liquid medium is a polar protic solvent or a mixture of polar protic solvents. Preferably the liquid medium is water.

In one embodiment the cationization reaction takes place in an inert liquid medium.

In one embodiment the cationized saccharide is subjected to an additional cationization.

In a second aspect, the present invention provides a derivatized saccharide. More particularly the present invention provides a derivatized saccharide, wherein the saccharide is derivatized with a compound having a conjugated electron withdrawing group.

In one embodiment the derivatized saccharide is produced with the method of the present invention via oxa-Michael addition reaction of a saccharide and a compound having a conjugated electron withdrawing group.

In one embodiment the saccharide comprises monosaccharides, disaccharides, oligosaccharides, polysaccharides or a mixture thereof.

In one embodiment the monosaccharide comprises glucose, fructose, galactose, mannose or a mixture thereof.

In one embodiment the disaccharide comprises sucrose, lactose, maltose, sucrose or a mixture thereof.

In one embodiment the oligosaccharide comprises glycan, raffinose, maltodextrin, cellodextrin or a mixture thereof.

In one embodiment the polysaccharide comprises starch, glycogen, galactogen, cellulose, chitosan, chitin, guar gum, pectin, dextran, α-glucan, cyclodextrin such as β-cyclodextrin or a mixture thereof.

In one embodiment the saccharide is glucose or cellulose.

In one embodiment the compound having a conjugated electron withdrawing group (EWG) comprises compounds having formula R1R2C═CR3-EWG, wherein R1, R2 and R3 represents independently a hydrogen atom or an alkyl chain having 1 to 20 carbon atoms; and EWG represents an electron withdrawing group, preferably a halogen, a nitrile, a carbonyl or a nitro group.

In one embodiment the R1, R2 and R3 represents independently a hydrogen atom or an alkyl chain having 1 to 10 carbon atoms

In one embodiment the compound having a conjugated electron withdrawing group is cationic.

In one embodiment the compound having a conjugated electron withdrawing group is selected from one of the following compounds I-X

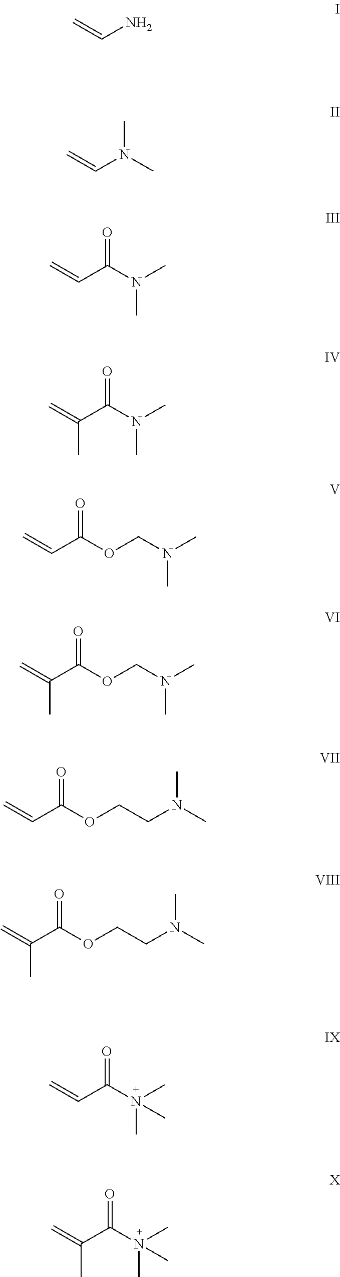

In one embodiment the compound having a conjugated electron withdrawing group is compound VII or VIII

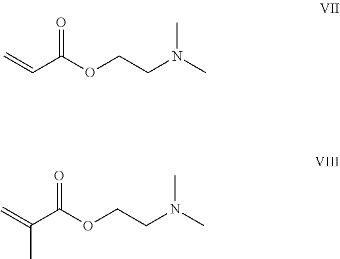

In one embodiment the derivatized saccharide is glucose or cellulose derivatized with compound VII or VIII

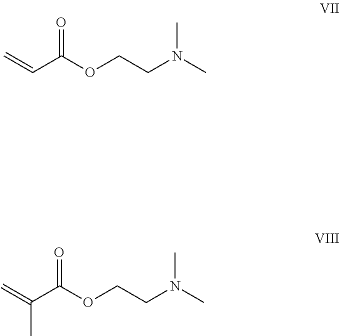

In one embodiment the compound having a conjugated electron withdrawing group is compound III or IV

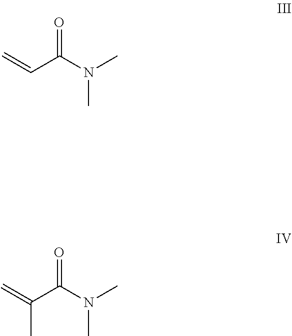

In one embodiment the compound having a conjugated electron withdrawing group is compound V or VI

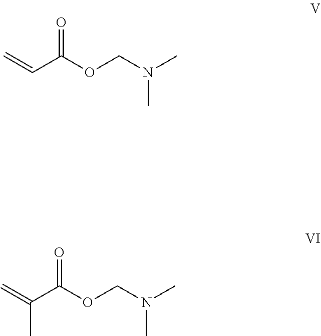

In one embodiment the compound having a conjugated electron withdrawing group is compound IX or X

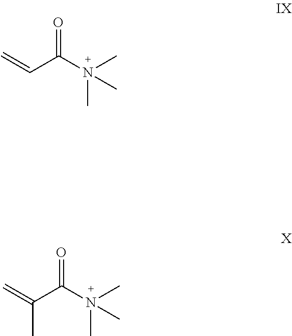

In one embodiment the compound having a conjugated electron withdrawing group is compound III, IV, V, VI, IX or X.

In one embodiment the compound having a conjugated electron withdrawing group is selected from one of the following compounds XXI-XXX

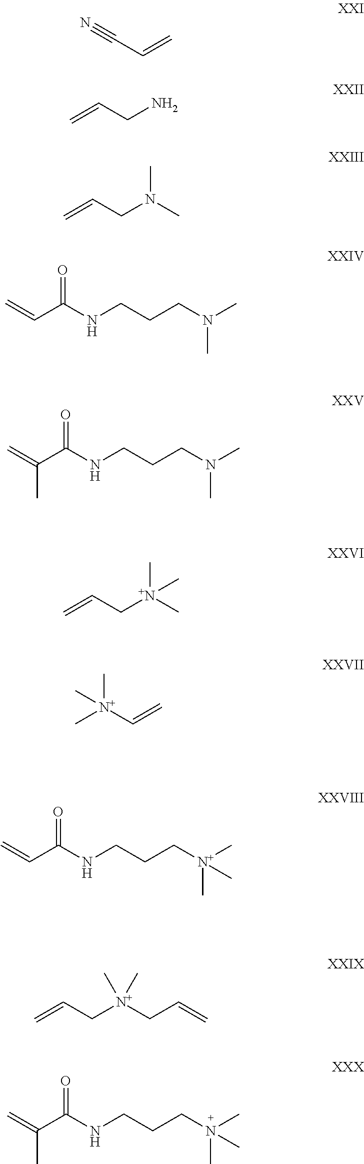

In one embodiment the derivatized saccharide has the following formula XI

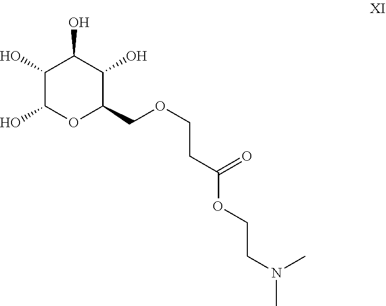 [SMILES: CN(C)CCOC(=O)CCOC[C@H]1O[C@H](O)[C@H](O)[C@@H](O)[C@@H]1O]

In a third aspect the present invention provides a cationic saccharide. The cationic saccharide comprises cationic saccharides having the following formula XII

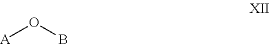

wherein A represents a monosaccharide, a disaccharide, an oligosaccharide or a polysaccharide; B represents a cationic compound having formula R1R2C—CR4R3-EWG, wherein R1, R2, R3 and R4 represents independently a hydrogen atom or an alkyl chain having 1 to 20 carbon atoms; EWG represents an electron withdrawing group, preferably a halogen, a nitrile, a carbonyl or a nitro group; and O having a bound to A is an oxygen atom of the monosaccharide, the disaccharide, the oligosaccharide or the polysaccharide.

In one embodiment the R1, R2, R3 and R4 represents independently a hydrogen atom or an alkyl chain having 1 to 10 carbon atoms.

In one embodiment the saccharide comprises monosaccharides, disaccharides, oligosaccharides, polysaccharides or a mixture thereof.

In one embodiment the monosaccharide comprises glucose, fructose, galactose, mannose or a mixture thereof.

In one embodiment the disaccharide comprises sucrose, lactose, maltose, sucrose or a mixture thereof.

In one embodiment the oligosaccharide comprises glycan, raffinose, maltodextrin, cellodextrin or a mixture thereof.

In one embodiment the polysaccharide comprises starch, glycogen, galactogen, cellulose, chitosan, chitin, guar gum, pectin, dextran, α-glucan, cyclodextrin such as β-cyclodextrin or a mixture thereof.

In one embodiment the cationic saccharide comprises cationic saccharides having the following formulas XIII-XX

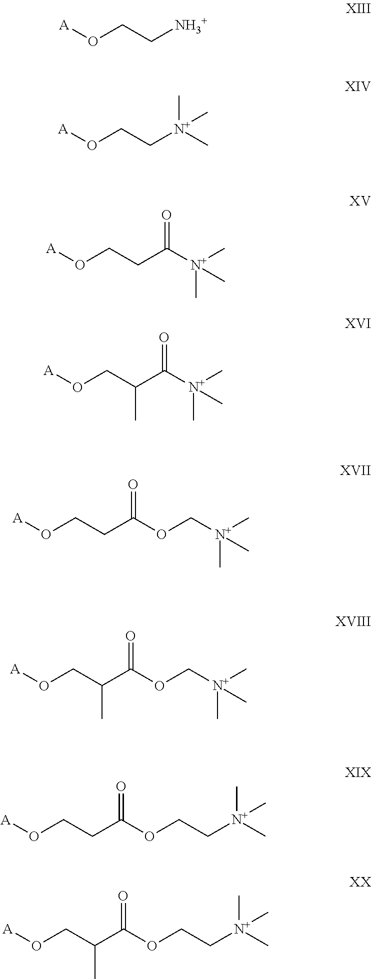

wherein A represents a monosaccharide, a disaccharide, an oligosaccharide or a polysaccharide and O having a bound to A is an oxygen atom of the monosaccharide, the disaccharide, the oligosaccharide or the polysaccharide.

In one embodiment the cationic saccharide has the following formula XIX or XX

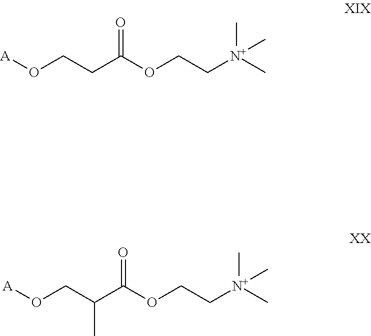

In one embodiment the cationic saccharide has the following formula XIX or XX

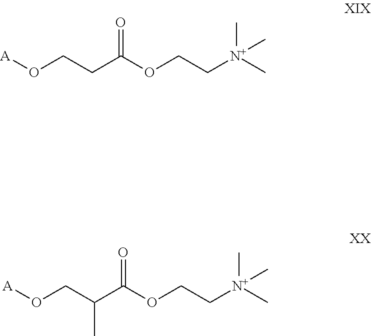

wherein A represents glucose or cellulose and O having a bound to A is an oxygen atom of the glucose or the cellulose.

In one embodiment the cationic saccharide has the following formula XV or XVI

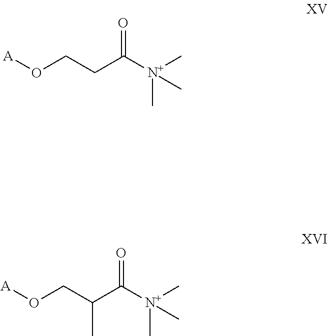

In one embodiment the cationic saccharide has the following formula XVII or XVIII

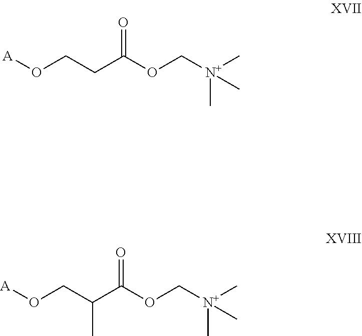

In one embodiment the cationic saccharide has formula XV, XVI, XVII or XVIII.

In one embodiment the cationic saccharide is produced with the method of the present invention.

In a fourth aspect the present invention provides use of the cationic saccharide.

More particularly, the present invention provides use of the cationic saccharide produced with the method of the present invention or the cationic saccharide of the present invention as a flocculant in industrial or municipal wastewater treatment, as a strength agent, a retention agent, a drainage agent or a sizing agent in papermaking, as a coagulant agent or as an additive in textile, cosmetic or paint industry.

### EXAMPLES

### Example 1 According to the Present Invention

### Materials

In table 1 is disclosed used compounds and properties of the compounds.

TABLE 1
Compounds and properties the compounds.

| | CAS | M | n | m | mp | bp | Density | Volume | Refractive |
|---|---|---|---|---|---|---|---|---|---|
| Starting materials | number | (g/mol) | (mol) | (g) | (° C.) | (° C.) | (g/ml) | (ml) | index |
| Glucose | 14431-43-7 | 198.17 | 0.0252 | 5.00 | 83 | | | | |
| (monohydrate) | | | | | | | | | |
| Dimethylaminoethyl | 2439-35-2 | 143.19 | 0.0250 | 3.58 | | 64 | 0.943 | 3.80 | 1.438 |
| acrylate | | | | | | | | | |
| NaOH | 1310-73-2 | 40.00 | 0.0010 | 0.04 | | | 2.13 | | |

### Synthesis of a-d-glucose (C6-OH) Derivatized with 2-(dimethylamino)ethyl acrylate

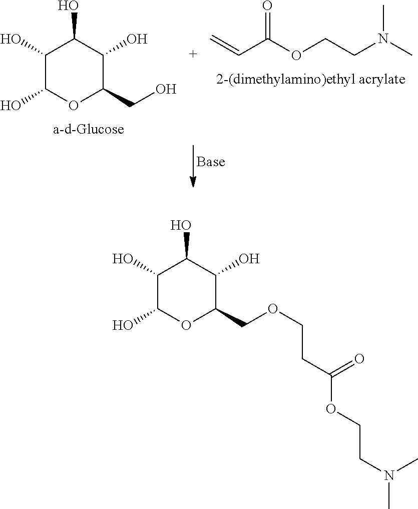

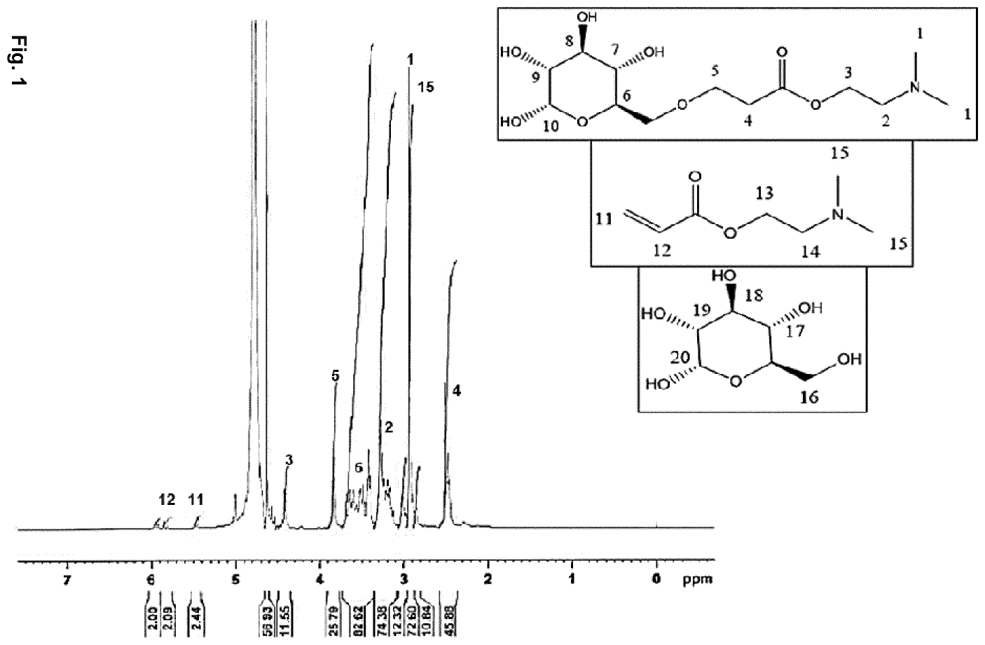

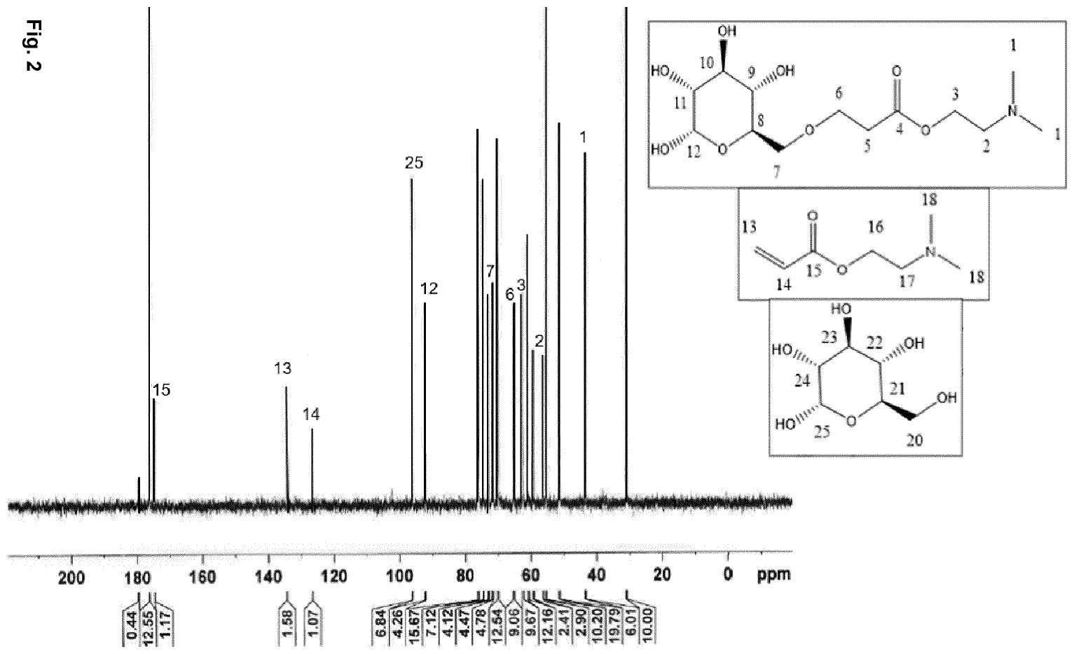

Glucose(monohydrate) was dissolved into 7 ml of pure water (MQ) followed by adding 0.04 g of NaOH. Reaction mixture was mixed with magnetic stirrer and 3.80 ml of dimethylaminoethyl acrylate (ADAM) was added, during mixing, dropwise during five minutes to the reaction mixture followed by mixing the reaction mixture for twelve hours. 1HNMR and 13CNMR profiles of the reaction mixture were analysed and molecular mass was measured after the 12 hours. The NMR profiles are shown in FIGS. 1 and 2.

### Characterisation

Molecular mass of the obtained a-d-glucose (C6-OH) derivatized with 2-(dimethylamino)ethyl acrylate is 323.34 g/mol.

FIG. 1 shows 1HNMR profile of the reaction mixture comprising the starting materials glucose(monohydrate) and dimethylaminoethyl acrylate and the product a-d-glucose (C6-OH) derivatized with 2-(dimethylamino)ethyl acrylate.

In the FIG. 1 carbons of the starting materials and the product are numbered. Chemical shifts of the protons having a bound to the numbered carbons are marked to the 1HNMR profile. Chemical shifts of protons of the glucose and the glucose unit are not marked to the 1HNMR profile except chemical shift of the proton having a bound to carbon 6.

FIG. 2 shows 13CNMR profile of a reaction mixture comprising the starting materials glucose(monohydrate) and dimethylaminoethyl acrylate and the product a-d-glucose (C6-OH) derivatized with 2-(dimethylamino)ethyl acrylate.

In the FIG. 2 carbons of the starting materials and the product are numbered. Chemical shifts of the carbons are marked to the 13CNMR profile. Chemical shifts of carbons of the glucose and the glucose unit are not marked to the 13CNMR profile except chemical shifts of the carbons 12 and 25.

Based on the measured molecular mass and the 1HNMR and 13CNMR profiles a derivatized saccharide, namely, a-d-glucose (C6-OH) derivatized with 2-(dimethylamino)ethyl acrylate is produced with the method of the present invention.

Various embodiments have been presented. It should be appreciated that in this document, words comprise, include, and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. A method for producing cationic saccharides, comprising reacting saccharide with a compound having a conjugated electron withdrawing group via oxa-Michael addition reaction in presence of a base or mixture of bases for producing a derivatized saccharide; and cationizing the derivatized saccharide.

2. The method according to claim 1, wherein one or more catalysts are present in the oxa-Michael addition reaction and/or in the cationization.

3. The method according to claim 1, wherein the derivatized saccharide is recovered prior cationization and the recovered derivatized saccharide is cationized.

4. The method according to claim 1, wherein the saccharide comprises monosaccharides, disaccharides, oligosaccharides, polysaccharides or a mixture thereof.

5. The method according to claim 1, wherein the compound having a conjugated electron withdrawing group (EWG) comprises compounds having formula R1R2C—CR3-EWG, wherein R1, R2 and R3 represents independently a hydrogen atom or an alkyl chain having 1 to 20 carbon atoms, preferably the alkyl chain has 1 to 10 carbon atoms; and EWG represents an electron withdrawing group, preferably a halogen, a nitrile, a carbonyl or a nitro group.

6. The method according to claim 1, wherein the compound having a conjugated electron withdrawing group is cationic.

7. The method according to claim 1, wherein the compound having a conjugated electron withdrawing group is selected from one of the following compounds I-X

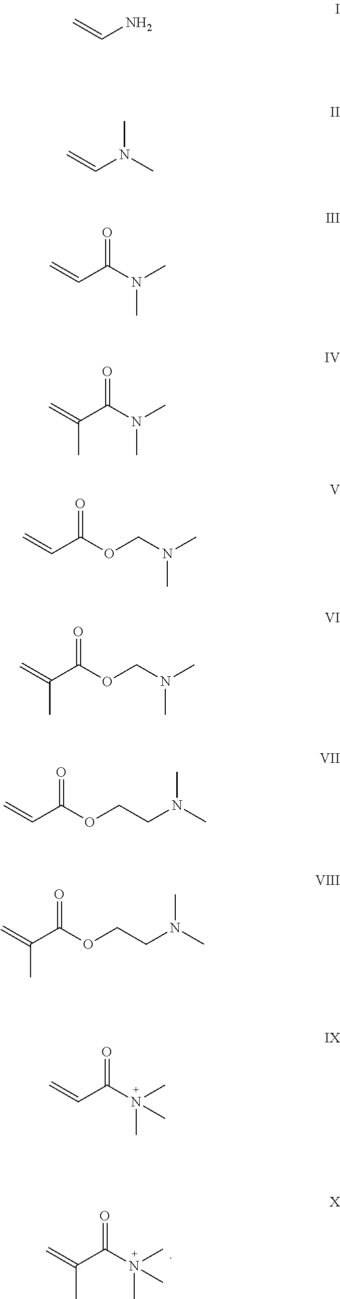

8. The method according to claim 1, wherein the compound having a conjugated electron withdrawing group is selected from one of the following compounds XXI-XXX

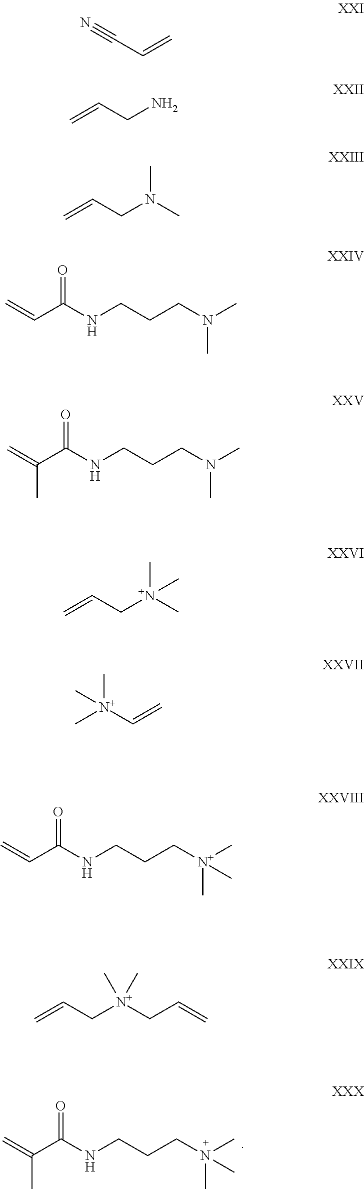

9. The method according to claim 1, wherein the compound having a conjugated electron withdrawing group is one of following compounds VII or VII

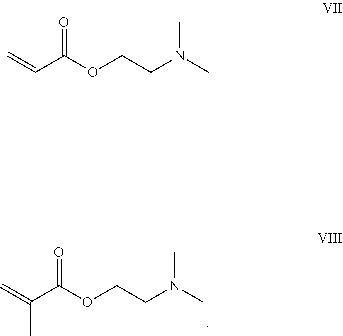

10. The method according to claim 1, wherein the compound having a conjugated electron withdrawing group is compound III, IV, V, VI, IX or X.

11. A derivatized saccharide comprising glucose or cellulose derivatized with a compound selected from compounds VII or VII

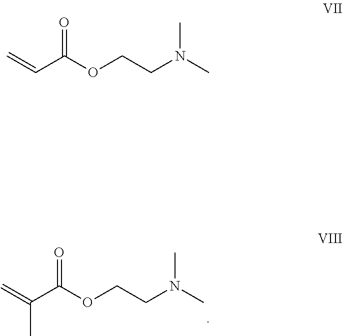

12. The derivatized saccharide according to claim 11, wherein the derivatized saccharide has the following formula XI

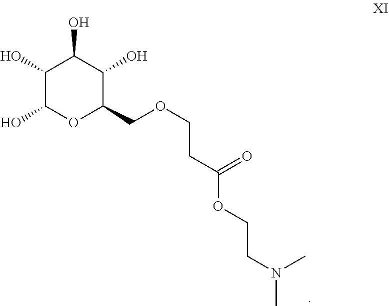 [SMILES: CN(C)CCOC(=O)CCOC[C@H]1O[C@H](O)[C@H](O)[C@@H](O)[C@@H]1O]

13. A cationic saccharide comprising cationic saccharides having the following formula XII 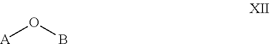
wherein A represents a monosaccharide, a disaccharide, an oligosaccharide or a polysaccharide; B represents a cationic compound having formula R1R2C—CR4R3-EWG, wherein R1, R2, R3 and R4 represents independently a hydrogen atom or an alkyl chain having 1 to 20 carbon atoms, preferably the alkyl chain has 1 to 10 carbon atoms; EWG represents an electron withdrawing group, preferably a halogen, a nitrile, a carbonyl or a nitro group; and O having a bound to A is an oxygen atom of the monosaccharide, the disaccharide, the oligosaccharide or the polysaccharide.

14. A cationic saccharide according to claim 13, wherein the cationic saccharide comprises cationic saccharides having the following formulas XIII-XX

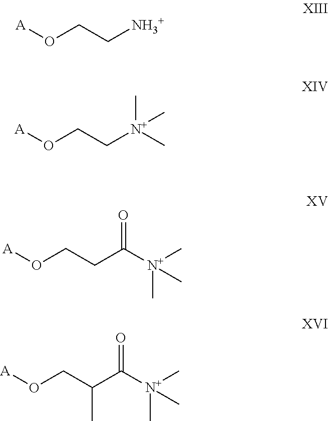

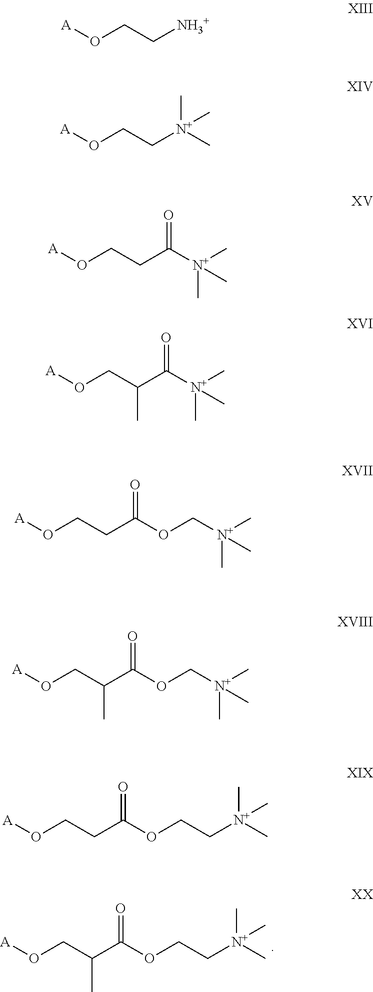

15. A cationic saccharide according to claim 13, wherein the cationic saccharide has the following formula XIX or XX

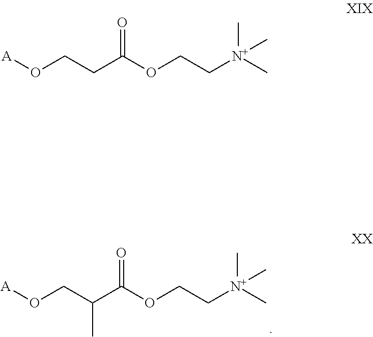

16. A cationic saccharide according to claim 13, wherein the cationic saccharide has formula XV, XVI, XVII or XVIII.

17. A cationic saccharide according to claim 13, wherein the A represents glucose or cellulose.

18. Use of the cationic saccharide produced with the method according to claim 1 or the cationic saccharide according to claim 13 as a flocculant in industrial or municipal wastewater treatment, as a strength agent, a retention agent, a drainage agent or a sizing agent in papermaking, as a coagulant agent or as an additive in textile, cosmetic or paint industry.
